(19) United States
(12) Patent Application Publication
(10) Pub. No.: US 2007/0269466 A1
(43) Pub. Date: Nov. 22, 2007

(54) Metal Oxide Particles Useful as Free Radical Scavengers in Sunscreen and Cosmetic Compositions

(75) Inventors: Gareth Wakefield, Oxford (GB); George Barry Park, Oxfordshire (GB)

Correspondence Address: WOLF GREENFIELD & SACKS, P.C., 600 ATLANTIC AVENUE, BOSTON, MA, 02210-2206, US

(73) Assignee: OXONICA LIMITED UNIT 7, KIDLINGTON OXFORD (GB)

(21) Appl. No.: 10/587,549

(22) PCT Filed: Jan. 27, 2005
(86) PCT No.: PCT/GB05/00262

§ 371 Date: Apr. 9, 2007

(30) Foreign Application Priority Data
Jan. 28, 2004 (GB) 0402832.1

(51) Int. Cl. A61K 8/49; C08K 3/22; A61K 8/37

(52) U.S. Cl. 424/401; 106/401; 106/453; 106/456; 106/479; 252/182.33; 424/59; 428/339; 430/270.1; 430/289.1; 504/367; 514/769; 524/403; 524/407; 524/408; 524/413; 524/431

(57) ABSTRACT

A composition is described which comprises a component which is susceptible to free radical attack, said composition comprising, as a free radical scavenger, at least one particle having a size not exceeding 100 nm of an oxide of a rare earth or transition metal possessing adjacent oxidation states. The composition is preferable a UV sunscreen composition suitable also for cosmetic use.

The present invention relates to metal oxide particles which are useful as free radical scavengers.

Many compounds are attacked by free radicals. Such compounds may be intrinsically stable; this attack is related to the homolytic bond dissociation energy. Such components include large molecules such as polymers as well as small molecules such as those with ethylenic unsaturation or those which possess a labile hydrogen atom, for example a tertiary hydrogen atom or other labile species including chlorine. Free radical attack may also break amide or ester linkages of small molecules or of large molecules such as polyamides or polyesters. These include organic sunscreen agents i.e organic compounds which absorb UV light. Attack can often be initiated by light, particularly UV light, but also gamma rays, X-rays and high energy electrons and/or heat. Once a free radical has been generated, a chain reaction can ensue essentially involving the formation of other free radicals in the molecule. Alternatively a free radical transfer reaction takes place whereby the free radical initially generated transfers to another molecule to initiate, or catalyse, free radical generation there. These mechanisms are, of course, well known to those skilled in the art. In polymer technology it is usual to incorporate antioxidants in the formulation, to protect the polymer both during its production which usually involves quite high temperatures (say 250°-300° C.) and also during its subsequent use where light is the usual activator of free radicals. It must be appreciated, therefore, that different antioxidants are frequently needed to cover these two eventualities.

It has surprisingly been found, according to the present invention, that the attack on any compound can be mitigated if the composition containing it contains small particles of certain oxides of transition metals or rare earth metals. Accordingly the present invention provides a composition which comprises a component which is susceptible to free radical attack, said composition comprising, as a free radical scavenger, at least one particle having a size not exceeding 100 nm of an oxide of a rare earth or transition metal possessing adjacent oxidation states.

Whether or not free radical attack occurs, and under what conditions, will in general be clear to the person skilled in the art in the given context. Whether or not the attack is adverse, and the conditions that are relevant, might well be different for different products and for different end uses. For example, for sunscreens and other compositions for cosmetic and topical use on the body, one might be concerned with adverse effects to ingredients that arise when the composition is subjected for say 8 hours to UV light of a wavelength from 290 to 400 nm at an intensity corresponding to midday Mediterranean sunlight, or say an intensity of 10 mW per square cm. In the case of compositions that are not used on the body, such as paints and coatings, adverse effects on ingredients that become apparent only after exposure of the composition over longer periods of time (for example one week, one month or one year), or under harsher conditions, might well be of concern. In preferred embodiments we are concerned with ingredients that undergo free radical attack.

The oxides which are used in the present invention are of rare earth metals or transition metals, preferably the latter. A “transition metal” as used herein is a member of group VA to VIII of the Periodic Table (see Chemistry of the Elements, Greenwood & Earnshaw, Pergamon 1984). The metals must possess adjacent oxidation states e.g. M²⁺ and M³⁺. Further it is generally necessary that these adjacent energy states differ by not more than 2 eV, preferably not more than 1 eV. Rare earth metals which can be used include terbium, europium and cerium which is preferred. Preferred transition metals which can be used include manganese, which is especially preferred, chromium, iron and vanadium as well as ruthenium; thus the preferred oxide is manganese oxide (MnO).

Although it will be more usual to employ a simple oxide, it is also possible to use mixed oxides. These mixed oxides will generally be of 2 or more transition/rare earth metals but the use of other metals such as aluminium and titanium is not excluded.

The particles used in the present invention can be obtained in a conventional manner. Thus, the particles may be prepared by controlled precipitation, combustion synthesis or flame pyrolysis as well as by other methods described in the literature for the production of nanoparticles.

In one embodiment of the present invention the particles are provided on an inert carrier, typically as a large particle, preferably of size not exceeding 10 microns and especially not exceeding 1 micron. Suitable carrier materials include inorganic oxides and compounds with oxyanions of elements such as aluminium, zirconium and silicon, especially silica or, for example, aluminium silicate. Thus the particles used in the present invention can “speckle” the surface of the layer carrier particle. By this means, the amount of the particles used in the present invention can more readily be determined. It is also possible to use the inert core material to influence other properties of the material. For example, in a plastics material the refractive index and mechanical properties may be altered by the core particle while the degradation is minimised by the nanoparticle-covered surface. In an emulsion the colour of the formulation may be controlled by light scattering from the core particle whilst scavenging efficiency is controlled by the surface.

Such particles can be prepared by decorating the core particles by electrostatic interaction before bonding the two phases together. For example, silica can be prepared by hydrolysis of tetraethyl orthosilicate (TEOS) by standard methods. The silica surface is negatively charged by attachment of a polyelectrolyte material, and precipitated nanoparticles of the free radical scavenging material are attached by electrostatic interaction. A further thin (generally less than 2 nm) silica layer then binds the phases together.

The presence of the particles in the composition results in free radical being trapped i.e. the composition is stabilised against the adverse effects of free radicals.

The present invention has particular applicability to compositions which contain titanium dioxide or zinc oxide since it is known that, on exposure to light, they generate free radicals. Titanium dioxide and zinc oxide absorb UV light efficiently, leading via the initial formation of electron hole pairs to the formation of superoxide and hydroxyl radicals. The crystalline forms of TiO₂, anatase and rutile, are semiconductors with band gap energies of about 3.23 and 3.06 eV respectively, corresponding to light of about 385 nm and 400 nm (1 eV corresponds to 8066 cm⁻¹). Thus these oxides while providing good wavelength cover can enhance the degradation of compounds including organic sunscreen agents, including UVA organic sunscreens, for example oxybenzene as well as cause degradation of other components of formulations. Attempts have been made to reduce the adverse effects of TiO₂ and ZnO by coating, but coatings are not invariably effective. Thus the present invention is particularly useful for paints, coatings and sunscreens which contain TiO₂ and/or ZnO. However, it is to be understood that the invention applies to all compositions which contain a component which is susceptible to free radical attack.

The presence of TiO₂ or ZnO or other activator may result in a change in a physical property of the component. With a polymer this may be, for example, a change in tensile strength or elongation at break; while with a small molecule, the free radical attack generally results in a change in its chemical structure which gives rise to a change in physical properties such as melting point, boiling point, viscosity, a change in its functional character or, in some cases, toxicity. All these changes can, of course, be measured as one skilled in the art would appreciate.

The present invention has particular applicability for UV sunscreen compositions suitable for cosmetic or pharmaceutical use. By “UV sunscreen composition suitable for cosmetic or topical pharmaceutical use” is meant any cosmetic or topical pharmaceutical composition having a component, generally an organic sunscreen agent which is susceptible to free radical attack, but it includes compositions whose principal function may not be sunscreening.

The average primary particle size of the particles should not exceed about 100 nm, preferably not exceed 50 nm, especially not exceed 20 nm and particularly not exceed 10 nm; there is no lower limit although for practical purposes it is about 1 nm. A desirable size in range is 30 to 5 nm, typically 15 to 5 nm. Since the scavenging effect is believed to be essentially catalytic it is desirable that the particles are as small as possible to maximise their surface area.

The oxide particles used in the present invention may have an inorganic or organic coating. For example, the particles may be coated with oxides of elements such as aluminium, zirconium or silicon, especially silica or, for example, aluminium silicate. The particles of metal oxide may also be coated with one or more organic materials such as polyols, amines, alkanolamines, polymeric organic silicon compounds, for example, RSi[{OSi(Me)₂}xOR¹]₃ where R is C₁-C₁₀ alkyl, R¹ is methyl or ethyl and x is an integer of from 4 to 12, hydrophilic polymers such as polyacrylamide, polyacrylic acid, carboxymethyl cellulose and xanthan gum or surfactants such as, for example, TOPO. If desired the surface doping can be carried out by a coating technique either separately or in combination with the inorganic or organic coating agent. Thus for example the undoped oxide can be coated with, say, manganese oxide along with an organic or inorganic coating agent such as silica. It is generally unnecessary to coat the oxide particles to render them hydrophilic so that for the aqueous phase the particles can be uncoated. However if the particles are to be in the organic or oily phase their surface needs to be rendered hydrophobic or oil-dispersible. This can be achieved by the application directly of, for example, a suitable hydrophobic polymer or indirectly by the application of a coating, for example of an oxide such as silica (which imparts a hydrophilic property) to which a hydrophobic molecule such as a metal soap or long chain (e.g. C₁₂-C₂₂) carboxylic acid or a metal salt thereof such as stearic acid, a stearate, specifically aluminium stearate, aluminium laurate and zinc stearate.

It should be understood that the term “coating” is not to be construed as being limited to a complete covering. Indeed it is generally beneficial for the coating not to be complete since the coating can act as a barrier to the interaction of the free radicals with the dopant on or in the surface of the particle. Thus it is preferred that the coating should be discontinuous where maximum scavenging effect is desired. However it will be appreciated that dopant on the surface can still act to quench free radicals generated within the particle in which case the coating can be continuous. Since coatings of silanes and silicones which can be polymeric or short chain or monomeric silanes are generally continuous these are generally less preferred. Thus coatings with an inorganic oxide are generally preferred since they generally do not result in a complete coating on the surface of the particles.

Typical coating procedures include the deposition of silica by mixing alkali, such as ammonium hydroxide, with an orthosilicate, such as TEOS, in the presence of the particle. Alternatively, the particle can first be coated with a silane such as (3-mercaptopropyl) trimethoxy silane (MPS), and then silicate, e.g. sodium silicate, is added. The silane attaches to the particle surface and acts as a substrate for the silicate, which then polymerises to form silica. Similar techniques can be used for other inorganic oxides.

The composition of the present invention, especially those containing a polymeric component, may be solid or liquid.

Typical solid materials include polymeric solids including three dimensional objects, films and fibres as well as textiles and fabrics e.g. clothing and netting made from woven and non-woven fibres as well as foamed articles. Three-dimensional objects include those made by melt-forming processes including extruded and moulded articles. Typical articles to which the present invention may be applied include generally external household and building materials including blinds and plastics curtains, trellis, pipes and guttering, cladding and facings such as soffit board and plastics roofing material which can be profiled as with corrugated sheeting, doors and windows frames. Other articles include advertising hoardings and the like e.g. advertising boards on vehicle sides as well as vehicle bodies and body parts including bumpers for cars, buses and trucks as well as roofs which can be used also for boats, as well as superstructures and hulls for boats and also bodies for lawnmowers and tractors and yachts, along with containers such as bottles, cans, drums, buckets and oil and water storage containers. Other objects include garden furniture.

Films to which the present invention can be applied include self supporting as well as non-self supporting films such as coatings. Self-supporting films to which the present invention applies include photographic films, packaging film and plastic film bearing indicia, typically as advertising film, which can also be applied over advertising hoardings. Such films can contain one or more customary ingredients for such products. Thus photographic film will contain one or more dyes or dye couplers and, optionally, a silver halide.

In some instances the polymeric composition itself is not liable to degradation but the composition is intended to protect a substrate or, in the case of a container, something placed in it. Thus such compositions can contain the metal oxide. Examples include pigmented and non-pigmented containers, typically bottles. Accordingly, the present invention also provides a self-supporting polymer composition, or a varnish composition, intended to protect a composition adjacent thereto from the adverse effects of light which comprises the specified metal oxide. In one embodiment the composition is three-dimensional and comprises a surface layer with the metal oxide while the non-surface part is generally not wood or a reconstituted wood such as chipboard, plywood or fibreboard and is preferably synthetic.

Coating compositions are typically paints and varnishes which contain a polymer either as the active ingredient as in some varnishes or as a support as in paints along with furniture polishes, waxes and creams; they can be aqueous or non aqueous i.e. contain an organic solvent. This coating composition can be in the form of a waterproofing agent. These coating compositions can contain one or more customary ingredients for such products. Some cosmetics compositions contain one or more polymers; such compositions are less preferred in the present invention.

The polymers which can be used in the compositions of the present invention include natural and synthetic polymers which may be thermoplastic or thermosetting.

The suitable polymers which may be homopolymers or copolymers which can be random, block or graft copolymers; the polymers can be crosslinked. Such polymers may be saturated or unsaturated. Typical polymers include alkylene polymers such as ethylene and propylene polymers, typically homopolymers, including polyethylene foams, including PTFE, siloxane and sulphide polymers, polyamides such as nylon, polyesters, acrylate and methacrylate polymers e.g. poly(methyl methacrylate) as well as PET, polyurethanes, including foams, vinyl polymers such as styrene polymers e.g. ABS, including polystyrene foam vinyl chloride polymers and polyvinyl alcohol. Fluorinated polymers such as PTFE and polyvinylidene fluoride can be used. Engineering polymers such as polyketones, for example PEEK, may also be used. The polymers can be thermosetting as with epoxy resins as well as phenolic, urea, melamine and polyester resins

Natural polymers which can be used include cellulosic polymers, as in paper including starch, polysaccharides, lignins, and polyisoprenes such as natural rubbers.

It will be appreciated that some polymers can be regarded as photostable in that there is no, or no significant, change in physical characteristics on exposure to UV light. These polymers are, therefore, not photosensitive and their exclusive use does not fall within the scope of the present invention.

Typical polymers for different applications include the following: (a) polyester, polyamide e.g. nylon, acrylics for fibres and fabrics; (b) polyester, polyvinyl chloride, polyethylene, polypropylene for bottles and the like; (c) polyethylene, polypropylene, polyvinyl chloride for film (non active such as packaging).

The liquid compositions of the present invention can be single phase, either aqueous or oily or multiphase, typically two-phase being oil-in-water or water-in-oil formulations. For single phase compositions the oxide particles should of course be dispersible in that phase. Thus the particles are desirably hydrophilic if the composition is aqueous or hydrophobic if the composition is oil-based. For two or multi-phase compositions the particles should be present in the phase containing the ingredient (or one of those ingredients) to be protected. It can, though, be desirable for the particles to be present in both aqueous and oily phases even if no ingredients which are to be protected are present in one of those phases. This can cover the situation where application of the composition by the user results in some phase transfer of the ingredient(s) to be protected. Also when an emulsion is spread on the skin it has a tendency to break down into oily and non-oily areas. When the water evaporates the oil-dispersible particles will tend to be in the oily areas thus leaving areas unprotected. This can be avoided by having both hydrophilic and hydrophobic particles in the emulsion so that some are retained in hydrophilic areas and others in hydrophobic areas. Desirably, the weight ratio of the water-dispersible particles to the oil-dispersible particles is from 1:4 to 4:1, preferably from 1:2 to 2:1 and ideally about equal weight proportions. Many organic suncreens are hydrophobic so that the particles should be hydrophobic but some organic suncreens by virtue of, in particular, acid groups are water soluble in which case the particles need to be hydrophilic in order to protect them.

The compositions of the present invention can be for cosmetics use and may be, for example, lipsticks, skin anti-ageing compositions in the form of, for example, creams, including anti-wrinkle formulation exfoliating preparations including scrubs, creams and lotions, skin lightening compositions in the form of, for example, face creams, preparations for the hands including creams and lotions, moisturising preparations, compositions for protecting the hair such as conditioners, shampoos and hair lacquers as well as hair masks and gels, skin cleansing compositions including wipes, lotions and gels, eye shadow and blushers, skin toners and serums as well as washing products such as shower gels, bath products including bubble baths, bath oils, but, preferably, sunscreens. In this connection we should point out that the expression “cosmetic UV sunscreening composition”, as used herein, includes any composition applied to the skin which may leave a residue on the skin such as some washing products. Compositions of the present invention may be employed as any conventional formulation providing protection from UV light. The composition may also be pharmaceutical compositions suitable for topical application. Such compositions are useful, in particular, for patients suffering from disorders of the skin which are adversely affected by UV light such those giving rise to polymorphous light eruptions.

Organic sunscreen agents which can be used in the compositions of the present invention include any conventional sunscreen agent which gives protection against UV light while if there is no other photosensitive component the sunscreen agent is photosensitive and/or is degraded by another ingredient of the composition. Suitable sunscreen agents are listed in the IARC Handbook of Cancer Prevention, vol. 5, Sunscreens, published by the International Agency for Research on Cancer, Lyon, 2001 and include:
(a) Para-aminobenzoic acids (PABA), (UVB absorbers) esters and derivatives thereof, for example amyldimethyl-; ethyldihydroxypropyl-; ethylhexyl dimethyl-; ethyl-; glyceryl-; and 4-bis-(polyethoxy)-PABA.
(b) Cinnamates (UVB) especially esters including methyl cinnamate esters and methoxycinnamate esters such as octylmethoxy cinnamate, ethyl methoxycinnamate, especially 2-ethylhexyl para-methoxycinnamate, isoamyl p-methoxy cinnamate, or a mixture thereof with diisopropyl cinnamate, 2-ethoxyethyl-4-methoxycinnamate, DEA-methoxycinnamate (diethanolamine salt of para-methoxy hydroxycinnamate) or α,β-di-(para-methoxycinnamoyl)-α′-(2-ethylhexanoyl)-glycerin, as well as diisopropyl methylcinnamate;
(c) benzophenones (UVA) such as 2,4-dihydroxy-; 2-hydroxy-4-methoxy; 2,2′-dihydroxy-4,4′-dimethoxy-; 2,2′-dihydroxy-4-methoxy-;′2,2′,4,4′-tetrahydroxy-; and 2-hydroxy-4-methoxy-4′-methyl-benzophenones, benzenesulphonic acid and its sodium salt; sodium 2,2′-dihydroxy-4,4′-dimethoxy-5-sulphobenzophenone and oxybenzone
(d) dibenzoylmethanes (UVA) such as butyl methoxydibenzoyl methane,especially 4-tert-butyl-4′methoxydibenzoylmethane;
(e) 2-phenylbenzimidazole-5 sulfonic acid UVB and phenyldibenzimidazole sulfonic acid and their salts;
(f) alkyl-β,β-diphenylacrylates (UVB) for example alkyl α-cyano-β,β-diphenylacrylates such as octocrylene;
(g) triazines (UVB) such as 2,4,6-trianilino-(p-carbo-2-ethyl-hexyl-1-oxy)-1,3,5 triazine as well as octyl triazone e.g. ethylhexyltriazone and diethylhexyl butamido triazone.
(h) camphor derivatives (generally UVB) such as 4-methylbenzylidene and 3-benzylidene-camphor and terephthalylidene dicamphor sulphonic acid (UVA), benzylidene camphor sulphonic acid, camphor benzalkonium methosulphate and polyacrylamidomethyl benzylidene camphor;
(i) organic pigment sunscreening agents such as methylene bis-benzotriazole tetramethyl butylphenol;
(j) silicone based sunscreening agents such as dimethicodiethyl benzal malonate.
(k) salicylates (UVB) such as dipropylene glycol-; ethylene glycol-, ethylhexyl-, isopropylbenzyl-, methyl-, phenyl-, 3,3,5-trimethyl- and TEA-salicylate (compound of 2-hydroxybenzoic acid and 2,2′2″-nitrilotris (ethanol));
(l) anthranilates (UVA) such as menthyl anthranilate as well as bisymidazylate (UVA), dialkyl trioleate (UVB), 5-methyl-2 phenylbenzoxazole (UVB) and urocanic acid (UVB).

Some compounds are effective for both UVA and UVB. These include methylene bisbenzotriazolyl tetramethylbutyl-phenol and drometrizole trisiloxane (Mexoryl XL).

The organic sunscreen agent(s) are typically present in the compositions at a concentration from 0.1 to 20%, preferably 1 to 10%, and especially 2 to 5%, by weight based on the weight of the composition.

Such compositions may be in the form of, for example, lotions, typically with a viscosity of 4000 to 10,000 mPas, e.g. thickened lotions, gels, vesicular dispersions, creams, typically a fluid cream with a viscosity of 10,000 to 20,000 mPas or a cream of viscosity 20,000 to 100,000 mPas, milks, powders, solid sticks, and may be optionally packaged as aerosols and provided in the form of foams or sprays.

The compositions may contain any of the ingredients used in such formulations including fatty substances, organic solvents, silicones, thickeners, liquid and solid emollients, demulcents, other UVA, UVB or broad-band sunscreen agents, antifoaming agents, antioxidants such as butyl hydroxy toluene, buffers such as lactic acid with a base such as triethanolamine or sodium hydroxide, plant extracts such as Aloe vera, cornflower, witch hazel, elderflower and cucumber, activity enhancers, moisturizing agents, and humectants such as glycerol, sorbitol, 2-pyrrolidone-5-carboxylate, dibutylphthalate, gelatin and polyethylene glycol, perfumes, preservatives, such as para-hydroxy benzoate esters, surface-active agents, fillers and thickeners, sequesterants, anionic, cationic, nonionic or amphoteric polymers or mixtures thereof, propellants, alkalizing or acidifying agents, colorants and powders, including metal oxide pigments with a particle size of from 100 nm to 20000 nm such as iron oxides along with conventional (undoped) TiO₂ and ZnO.

It is known that other ingredients of cosmetic compositions, for example some surface-active agents may have the effect of degrading certain sunscreen agents in the presence of UV light. Also TiO₂ and ZnO are known to degrade certain organic sunscreens such as avobenzone as well as antioxidants such as vitamins e.g. vitamins A, B, C and E. It will be appreciated that it is particularly useful to use the doped TiO₂ and/or ZnO and/or reduced ZnO with such sunscreens. This is because TiO₂ and ZnO do generally have a positive UV absorptive effect. Thus by using the doped TiO₂ and/or ZnO and/or reduced ZnO it may be possible to use less antioxidant or make the formulation longer lasting.

The organic solvents are typically from lower alcohols and polyols such as ethanol, isopropanol, propylene glycol, glycerin and sorbitol as well as methylene chloride, acetone, ethylene glycol monoethyl ether, diethylene glycol monobutyl ether, diethylene glycol mono-ethyl, ether, dimethyl sulphoxide, dimethyl formamide and tetrahydrofuran.

The fatty substances may consist of an oil or wax or mixture thereof, fatty acids, fatty acid esters, fatty alcohols, Vaseline, paraffin, lanolin, hydrogenated lanolin or acetylated lanolin, beeswax, ozokerite wax and paraffin wax.

The oils are typically from animal, vegetable, mineral or synthetic oils and especially hydrogenated palm oil, hydrogenated castor oil, Vaseline oil, paraffin oil, Purcellin oil, silicone oil such as polydimethyl siloxanes and isoparaffin.

The waxes are typically animal, fossil, vegetable, mineral or synthetic waxes. Such waxes include beeswax, Carnauba, Candelilla, sugar cane or Japan waxes, ozokerites, Montan wax, microcrystalline waxes, paraffins or silicone waxes and resins.

The fatty acid esters are, for example, isopropyl myristate, isopropyl adipate, isopropyl palmitate, octyl palmitate, C₁₂-C₁₅ fatty alcohol benzoates (“FINSOLV TN” from FINETEX), oxypropylenated myristic alcohol containing 3 moles of propylene oxide (“WITCONOL APM” from WITCO), capric and caprylic acid triglycerides (“MIGLYOL 812” from HULS).

The compositions may also contain thickeners such as cross-linked or non cross-linked acrylic acid polymers, and particularly polyacrylic acids which are cross-linked using a polyfunctional agent, such as the products sold under the name “CARBOPOL” by the company GOODRICH, cellulose, derivatives such as methylcellulose, hydroxymethylcellulose, hydroxypropyl methylcellulose, sodium salts of carboxymethyl cellulose, or mixtures of cetylstearyl alcohol and oxyethylenated cetylstearyl alcohol containing 33 moles of ethylene oxide.

Suitable emollients include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, caster oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

Suitable propellants include propane, butane, isobutane, dimethyl ether, carbon dioxide, nitrous oxide.

Suitable powders include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

When the compositions of the present invention are sunscreens they may be in the form of, for example, suspensions or dispersions in solvents or fatty substances or as emulsions such as creams or milks, in the form of ointments, gels, solid sticks or aerosol foams. The emulsions, which can be oil-in-water or water-in-oil emulsions, may further contain an emulsifier including anionic, nonionic, cationic or amphoteric surface-active agents; for a water-in-oil emulsion the HLB is typically from 1 to 6 while a larger value i.e >6 is desirable for an oil-in-water emulsion. Generally water amounts to up to 80%, typically 5 to 80%, by volume. Specific emulsifiers which can be used include sorbitan trioleate, sorbitan tristearate, glycerol monooleate, glycerol monostearate, glycerol monolaurate, sorbitan sesquioleate, sorbitan monooleate, sorbitan monostearate, polyoxyethylene (2) stearyl ether, polyoxyethylene sorbitol beeswax derivative, PEG 200 dilaurate, sorbitan monopalmitate, polyoxyethylen (3.5) nonyl phenol, PEG 200 monostearate, sorbitan monostearate, sorbitan monolaurate, PEG 400 dioleate, polyoxyethylene (5) monostearate, polyoxyethyene (4) sorbitan monostearate, polyoxyethylene (4) lauryl ether, polyoxyethylene (5) sorbitan monooleate, PEG 300 monooleate, polyoxyethylene (20) sorbitan tristearate, polyoxyethylene (20) sorbitan trioleate, polyoxyethylene (8) monostearate, PEG 400 monooleate, PEG 400 monostearate, polyoxyethylene (10) monooleate, polyoxyethylene (10) stearyl ether, polyoxyethylene (10) cetyl ether, polyoxyethylene (9.3) octyl phenol, polyoxyethylene (4) sorbitan monolaurate, PEG 600 monooleate, PEG 1000 dilaurate, polyoxyethylene sorbitol lanolin derivative, polyoxyethylene (12) lauryl ether, PEG 1500 dioleate, polyoxyethylene (14) laurate, polyoxyethylene (20) sorbitan monostearate, polyoxyethylene (20) sorbitan monooleate, polyoxyethylene (20) stearyl ether, polyoxyethylene (20) sorbitan monopalmitate, polyoxyethylene (20) cetyl ether, polyoxyethylene (25) oxypropylene monostearate, polyoxyethylene (20) sorbitol monolaurate, polyoxyethylene (23) lauryl ether, polyoxyethylene (50) monostearate, and PEG 4000 monostearate. Alternatively the emulsifier can be silicone surfactant, especially a dimethyl polysiloxane with polyoxyethylene and/or polyoxypropylene side chains, typically with a molecular weight of 10,000 to 50,000, especially cyclo-methicone and dimethicone copolyol. They may also be provided in the form of vesicular dispersions of ionic or nonionic amphiphilic lipids prepared according to known processes.

The present invention is also applicable to any composition intended for agricultural or horticultural use which contains an organic active ingredient as well as to veterinary compositions containing an organic active ingredient, generally for topical application. Generally the active ingredient will be a biocide but it can be, for example, a plant growth promoter or regulator. Thus the compositions of the present invention may be herbicides, fungicides, insecticides, bactericides, acaricides, molluscicides, miticides or rodenticides, which can be broad spectrum or selective. The present invention is particularly useful for fast knockdown insecticides which are badly affected by UV light and free radicals. Veterinary compositions can take the form of, for example, antiseptic or wound healing preparations.

The compositions of the present invention can also be formulated for household use as with, for example, insecticides and rodenticides. Accordingly, the present invention also provides a composition suitable for household use which comprises at least one organic biocide and the metal oxide.

The compositions of the present invention can contain any of the organic active ingredients currently employed for such compositions.

Suitable herbicides which can be used in the present invention include triazines, amides, in particular haloacetanilides, carbamates, toluidines (dinitroanilines), ureas, plant growth hormones, in particular phenoxy acids and diphenyl ethers. Thus herbicides which may be used include phenoxy alkanoic acids, bipyridiniums, benzonitriles with phthalic compounds, dinitroanilines, acid amides, carbamates, thiocarbamates, heterocyclic nitrogen compounds including triazines, pyridines, pyridazinones, sulfonylureas, imidazoles and substituted ureas as well as halogenated aliphatic carboxylic acids, some inorganic and organic materials and derivatives of biologically important amino acids. Specific herbicides which can be used in the present invention include 2,4-dichlorophenoxyacetic acid (2,4-D) and 2, 4, 5-trichlorophenoxyacetic acid (2, 4, 5-T). Suitable triazines include 2-chloro-, 2-methylthio-, 2-methoxy-4,6-bis-(alkylamino)-s-triazines as well as some 2-azido-substituted triazines. Typical herbicidal ureas include monuron (3-p-chlorophenyl)-1,1-dimethylurea) as well as diuron, neburon, fenuron and chloroxuron. Suitable carbamates include N-phenylcarbamate and isopropyl carbanilate (propham) and substituted derivatives thereof including isopropyl m-chlorocarbanilate (chlorpropham) as well as barban, swep, dichlormate and terbutol. Suitable thiocarbamates include EPTC, metham, vernolate, CDEC, pebulate, diallate, triallate, butylate, molinate, cycloate, thiobencarb and ethiolate. Suitable amide herbicides include solan, dicryl, propanil, dipehamid, propachlor, alachlor, CDAA, naptalam, butachlor, prynachlor and napropamide. Suitable chlorinated aliphatic acids include triochloroacetic acid (TCA), dalapon and 2,2,3-trichloropropionic acid. Suitable chlorinated benzoic acids include chloramben, DCPA, dicamba, dichlobenil and 2,3,6-TBA. Phenolic herbicides which can be used include bromoxynil, ioxynil, DNOC and dinoseb. Suitable dinitroanilines which can be used include benefin, trifluralin, nitralin, oryzalin, isopropalin, dinitramine, fluchloralin, profluralin and butralin. Suitable bypyridinium herbicides include diquat and paraquat salts and derivatives thereof.

Suitable insecticides which can be used in the present invention include nicotinoids, rotenoids, derivatives of the seeds of sabadilla and the plant ryania speciosa and pyrethroids as well as organochlorine insecticides, organophosphorus insecticides, carbamate insecticides and various insect growth regulators.

Suitable nicotinoids include nicotine sulfate and imidocloprid. The pyrethroids constitute a large group of insecticides most of which are now synthetic including resmethrin, phenothrin, cyphenothrin, empenthrin, prallethrin, permethrin, cypermethrin, alpha cypermethrin, tetramethrin and delta tetramethrin, including their isomers, especially optical isomers along with derivatives of these. Suitable organochlorine insecticides include DDT (dichlorodiphenyltrichloroethane) along with methoxychlor and perthane, as well as lindane, toxaphene, chlordane, heptachlor, aldrin, dieldrin and endrin. Suitable organophosphorus insecticides include phosphoric acid and phosphorothioic acid anhydrides, aliphatic phosphorothioate esters along with phenyl phosphorothioate esters, phenyl phosphorodithioate esters, phosphonothioate esters of phenols, vinyl phosphates, phosphorothioate esters of heterocyclic enols and of s-methyl heterocycles. Of these specific mention can be made of parathion, methyl parathion, dicapthon, chlorthion, fenitrothion, fenthion and fensulfothion along with fenchlorphos, cyanophos, propafos and temephos. Suitable carbamate insecticides which can be used include carbaryl, carbofuran, propoxur, dioxacarb, bendiocarb, mexacarbate, isoprocarb and ethiofencarb. Suitable acaricides include chlorfenethol, chlorobenzilate, dicofol, tetradifon, sulphenone, ovex, propargite, cyhexatin and dienochlor.

Some of the insecticides given above are suitable for killing rodents but other rodenticides which can be used include acute rodenticides and chronic poisons include anticoagulants; these can be stomach poisons, contact poisons or fumigants. Such anticoagulants include dicoumarol, warfarin, coumatetraly, coumachlor, difenacoum, brodifacoum, bromadiolone, pindone, diphacinone and chlorophacinone.

Insecticides which can be used in the compositions of the present invention can also be in the form of microbial agents since insects are attacked by many pathogens. These include bacterial agents, in particular bacillus microorganisms, especially bacillus thuringiensis (b.t.) strains such as b.t. aizawa, israelensis, kurstaki and tenebrionis, fungal agents, protozoa and viruses.

Suitable fungicides which can be used in the compositions of the present invention include elements such as sulphur, copper, mercury and tin along with thiocarbamate and thiurame derivatives, phthalimides and trichloromethylthiocarboximides, aromatic hydrocarbons and dicarboximides. Specific examples include ferbam, ziram, thiram, zineb, maneb and mancozeb as well as dimethylthiocarbamates and ethylene bis-dithiocarbamates. Other useful fungisides include captan, folpet, captafol and dichlofluanid. Suitable aromatic hydrocarbons include quintozene, dinocap, chloroneb, dichloran, dichlone and chlorothalonil along with oxazolidinediones such as vinclozolin, chlozolinate, hydantoin such as iprodione and succinimide such as procymidone. Other fungicides which can be used include guanidine salts such as dodine, quinones such as dithianon, quinoxalines such as chinomethionat, pyridazines such as diclomezine, thiadiazoles such as etridiazole, pyrroles such as fenpiclonil, quinolines such as ethoxyquin and triazines such as anilazine. Other fungicides which can be used include mitochondrial respiration inhibitors which are generally carboxanilides including carbox, oxycarboxin, flutolanil, fenfuram, mepronil, methfuroxam and metsulfovax. Further fungicides which can be used include microtubuline polymerization inhibitors including thiabendazole, fuberidazole, carbendazim, benomyl and thiophanate methyl. Other suitable fungicides include inhibitors of sterol biosynthesis including C-14 demethylation inhibitors such as triazoles which have a 1,2,4-triazole group attached through the 1-nitrogen to a large lipophilic group, in particular triadimefon, propiconazole, tebuconazole, cyproconazole and tetraconazole along with flusilazole which incorporates a silicon atom, myclobutanil, flutriafol and imibenconazole. Other fungicides which can be used include RNA biosynthesis inhibitors, phospholipid biosynthesis inhibitors, melanin biosynthesis inhibitors, fungal protein biosynthesis inhibitors and cell wall biosynthesis inhibitors.

Such compositions can be in liquid or solid form. Liquid compositions can be aqueous or non aqueous while solid forms include powders or dusts, granules and tablets. For rodenticides, in particular, the compositions can take the form of a bait, especially a foodstuff, for example grain, which has been treated with the rodenticide and the special oxide.

The concentration of the active ingredient in the composition can vary within a wide range but is typically 0.5 to 95, for example 1 to 50% by weight.

A composition according to the invention preferably contains from 0.5% to 95% by weight (w/w) of active ingredient.

The compositions for agricultural or horticultural use according to the invention generally contain a carrier to facilitate application to the locus to be treated, which may for example be a plant, seed or soil, or to facilitate storage, transport or handling. The carrier may be a solid, or a liquid, as well as material which is normally a gas but which has been compressed to form a liquid.

The compositions may be in the form of, for example, emulsion concentrates, solutions, oil in water emulsions, wettable powders, soluble powders, suspension concentrates, dusts, granules, water dispersible granules, micro-capsules and gels. Other substances, such as fillers, solvents, solid carriers, surface active compounds (surfactants), and optionally solid and/or liquid auxiliaries and/or adjuvants can be present. The composition can be formulated for dispersing by, for example, spraying, atomizing, dispersing or pouring.

Solvents which may be used include aromatic hydrocarbons, e.g. substituted naphthylenes, phthalic acid esters such as dibutyl or dioctyl phthalate, aliphatic hydrocarbons, e.g. cyclohexane or paraffins, alcohols and glycols as well as their ethers and esters, e.g. ethanol, ethyleneglycol mono- and dimethyl ether, ketones such as cyclohexanone, strongly polar solvents such as N-methyl-2-pyrrolidone or γ-butyrolactone, higher alkyl pyrrolidones, e.g. n-octylpyrrolidone or cyclohexylpyrrolidone, epoxidized plant oil esters, e.g. methylated coconut or soybean oil ester and water. Mixtures can also be used.

Solid carriers, which may be used for dusts, wettable powders, water dispersible granules, or granules, include mineral fillers, such as silicas, calcite, talc, kaolin, montmorillonite or attapulgite. The physical properties may be improved by addition of highly dispersed silica gel or polymers. Carriers for granules may be porous material, e.g. pumice, kaolin, sepiolite, bentonite; non-sorptive carriers may be calcite or sand.

The compositions can be formulated as concentrates which can subsequently be diluted by the user before application. The presence of small amounts of a carrier which is a surfactant facilitates this process of dilution. Thus, preferably the compositions according to the invention preferably contain a surfactant. For example, the composition may contain two or more carriers, at least one of which is a surfactant. Such surfactants may be nonionic, anionic, cationic or zwitterionic.

The compositions of the invention may for example be formulated as wettable powders, water dispersible granules, dusts, granules, solutions, emulsifiable concentrates, emulsions, suspension concentrates and aerosols. Wettable powders usually contain 5 to 90% w/w of active ingredient and 3 to 10% w/w of dispersing and/or wetting agent and, where desirable, 0 to 10% w/w of stabilizer(s) and/or other additives such as penetrants or stickers. Dusts are usually formulated as a dust concentrate having a similar composition to that of a wettable powder but without a dispersant. Water dispersible granules are usually prepared to have a size from 0.15 mm to 2.0 mm and contain 0.5 to 90% w/w active ingredient and 0 to 20% w/w of additives such as stabilizers, surfactants, slow release modifiers and binding agents. Emulsifiable concentrates usually contain, in addition to a solvent or a mixture of solvents, 1 to 80% w/v active ingredient, 2 to 20% w/v emulsifiers and 0 to 20% w/v of other additives such as stabilizers, penetrants and corrosion inhibitors. Suspension concentrates usually contain 5 to 75% w/v active ingredient, 0.5 to 15% w/v of dispersing agents, 0.1 to 10% w/v of suspending agents such as protective colloids and thixotropic agents, 0 to 10% w/v of other additives such as defoamers, corrosion inhibitors, stabilizers, penetrants and stickers, and water or an organic liquid in which the active ingredient is substantially insoluble; certain organic solids or inorganic salts may be present dissolved in the formulation to assist in preventing sedimentation and crystallization or as antifreeze agents for water.

In general, the compositions can contain the usual additional ingredients characteristic for the composition in question including inorganic and organic pigments, including TiO₂ and/or ZnO, fillers and extenders as well as light stabilisers, typically hindered amine stabilisers.

In the compositions the metal oxides are preferably present at a concentration of about 0.5 to 20% by weight, preferably about 1 to 10% by weight and more preferably about 3 to 8% by weight, in particular about 4 to 7%, such as 4 to 6%, for example about 5% by weight.

1. A composition which comprises a component which is susceptible to free radical attack, said composition comprising, as a free radical scavenger, at least one particle having a size not exceeding 100 nm of an oxide of a rare earth or transition metal possessing adjacent oxidation states.

2. A composition according to claim 1 wherein the energy levels of said adjacent oxidation states do not differ by more than 2 eV.

3. A composition according to claim 1 wherein the oxide is of cerium, manganese, vanadium, chromium or iron.

4. A composition according to claim 3 wherein the oxide is manganese oxide.

5. A composition according to claim 1 wherein the oxide has a particle size not exceeding 20 nm.

6. A composition according to claim 5 wherein the oxide has a particle size from 5 to 15 nm.

7. A composition according to claim 1 wherein the oxide is present in an amount from 0.5 to 20% by weight.

8. A composition according to claim 1 which is a UV sunscreen composition.

9. A composition according to claim 1 which is suitable for cosmetic use.

10. A composition according to claim 8 which contains a W sunscreen agent which is adversely affected by free radicals.

11. A composition according to claim 8 wherein the organic sunscreen agent is a paraaminobenzoic acid, ester or derivative thereof, a methoxy cinnamate ester, a benzophenone, a dibenzylomethane, an allyl-β, p-phenyl acrylate, a triazine, a camphor derivative, an organic pigment, a silicone based sunscreen agent or 2-phenylbenzimdazoyl-5 sulphonic acid or phenyldibenzimidazoyl sulphonic acid.

12. A composition according to claim 8 which is a sunscreen.

13. A composition according to claim 8 which is in the form of a lotion, gel, dispersion, cream, milk, powder or solid stick.

14. A composition according to claim 12 which comprises a water-dispersible and an oil-dispersible metal oxide.

15. A composition according to claim 1 which is a polymeric composition.

16. A composition according to claim 15 wherein the polymeric material is thermoplastic.

17. A composition according to claim 15 wherein the polymeric material is thermosetting.

18. A composition according to claim 15 which is in the form of a three dimensional article.

19. A composition according to claim 15 which is in the form of a film or a fibre or a fabric.

20. A composition according to claim 19 which is in the form of a photographic film.

21. A composition according to claim 15 which is in the form of a coating composition.

22. A composition according to claim 15 which is in the form of a paint or varnish.

23. A composition according to claim 1 which comprises at least one organic veterinarally, agriculturally and/or horticulturally active compound.

24. A composition according to claim 1 suitable for household use which comprises at least one organic biocide.

25. A composition according to claim 23 wherein the active compound is a herbicide, fungicide, insecticide, acaricide, miticide or rodenticide.

26. (canceled)

27. A method for reducing the concentration of one or more components susceptible to free radical attack in a composition containing said component comprising incorporating into the composition a particle of a metal oxide as defined in claim 1 to reduce the concentration of the one or more components susceptible to free radical attack in the composition.

28. A method for reducing the rate of loss in UV absorption of a sunscreen composition containing an organic UV sunscreen agent which is adversely affected by free radical attack comprising incorporating into the composition a particle of a metal oxide as defined in claim 1 to reduce the rate of loss in UV absorption of the sunscreen composition containing the organic UV sunscreen agent which is adversely affected by free radical attack.

29. A method for increasing the shelf life of one or more veterinarally, agriculturally and/or horticulturally active compounds in a composition suitable for veterinary, agricultural, horticultural or household use comprising incorporating into the composition a particle of a metal oxide as defined in claim 1 to increase the shelf life of the one or more veterinarally, agriculturally and/or horticulturally active compounds in the composition suitable for veterinary, agricultural, horticultural or household use.

30. A method of increasing the effectiveness of a composition which comprises one or more components which are susceptible to free radical attack which comprises incorporating into the composition a particle of a metal oxide as defined in claim 1.
